(19) United States
(12) Patent Application Publication
(10) Pub. No.: US 2026/0294651 A1
(43) Pub. Date: Oct. 1, 2026

(54) METHOD OF DESIGNING AND MANUFACTURING A CUSTOMIZED HAND PROSTHESIS

(71) Applicants: UNIVERSITÀ CAMPUS BIO-MEDICO DI ROMA, Rome (IT); INAIL - CENTRO PROTESI, Rome (IT)

(72) Inventors: Nevio Luigi TAGLIAMONTE, Rome (IT); Alessandro CECCARELLI, Monterotondo RM (IT); Fabrizio TAFFONI, Sermoneta LT (IT); Francesca CORDELLA, Rome RM (IT); Loredana ZOLLO, Rome RM (IT); Martina LAPRESA, Ariccia RM (IT); Ludovica NINI, Rome RM (IT); Emanuele GRUPPIONI, Castel Maggiore BO (IT); Kavin MORELLATO, Bologna BO (IT)

(73) Assignees: UNIVERSITÀ CAMPUS BIO-MEDICO DI ROMA, Rome (IT); INAIL - CENTRO PROTESI, Rome (IT)

(21) Appl. No.: 19/478,478

(22) PCT Filed: Jan. 23, 2024
(86) PCT No.: PCT/IB2024/050641

§ 371 Date: Oct. 24, 2025

(30) Foreign Application Priority Data
Apr. 26, 2023 (IT) 102023000008163

(51) Int. Cl. A61F 2/50; A61B 5/107; A61B 5/11; A61F 2/58; B33Y 50/00; B33Y 80/00; G05B 19/4099

(52) U.S. Cl. CPC A61F 2/5046; A61B 5/1077; A61B 5/1122; B33Y 50/00; B33Y 80/00; G05B 19/4099; A61F 2002/505; A61F 2002/587; G05B 2219/35134

(57) ABSTRACT

A method for designing a functional prosthesis of customized hand, for patients amputated at trans-radial level, implemented with additive manufacturing technology. The method flow for designing and developing the devices consists of the following steps: i) kinematic analysis and 3D scan of the limb contralateral to the amputated one; ii) kinematic synthesis of the planar mechanism at one active degree of freedom (GdL), modular for each finger, and optimization of the same through a global performance index (GPI); iii) prototyping of the assembly of five fingers.

### TECHNICAL FIELD OF THE INVENTION

The present invention relates to a method for designing and manufacturing a customized hand prosthesis implemented at least partially by electronic computer. The method is particularly suitable for patients with unilateral trans-radial amputation.

### BACKGROUND

The hand, evidently, plays a function of primary importance in the activities of daily living (ADL). The long fingers (index finger, middle finger, ring finger, little finger) are formed by the metacarpophalangeal (MCP), proximal interphalangeal (PIP) and distal interphalangeal (DIP) joints. The thumb is formed by the carpometacarpal (CMC), metacarpophalangeal (MCP) and interphalangeal (IP) joints. Such joints assist a total of 21 degrees of freedom (GdL), which allow a very fine manipulation of the surrounding reality and of the objects composing it.

The level of disability caused by hand amputation was assessed to be equal even to 54% [K. D. Capek, B. D. Hughes, and G. D. Warden, “Functional sequelae and disability assessment,” in Total Burn Care: Fifth Edition, Elsevier Inc., 2017, pp. 673-679].

The prosthetic hands available in the state of art have different levels of complexity. In particular, they can consist in purely cosmetic systems or implement polyarticular and anthropomorphic robotic devices then capable not only to fill the aesthetic deficit, but also to provide for a functional replacement. The main parameter to be considered to evaluate the effectiveness of an anthropomorphic poly-articulated robotic prosthesis is the relationship between dexterity, that is the number of implementable grasps, and the weight, which should be maintained below the average one of an adult hand (about 600 g). However, such requirement of reduced weight is in conflict with the one related to the number of motions to be repeated, since a high dexterity requires the use of multiple independent actuators, with consequent increase in weight and overall dimension of the prosthesis.

Generally, indeed for the need of optimizing opposing technical needs, the poly-articulated prosthetic devices tend to result to be complex and expensive. Consequently, the access to such devices is very limited. Moreover, the poly-articulated solutions are often abandoned due to their excessive use complexity and, often, missing naturalness of appearance.

The prostheses provided in the art, then, result to be able still to be optimized with respect to the necessary compromise between anthropometric and functional customization, dexterity, ergonomics, aesthetics and cost.

In particular, the Inventors have identified the following four specific problems, the first two pertaining to the aesthetic field and the others to the functional one.
(a) The known systems use discrete solutions based upon sizes, which do not allow a satisfying adaptation to the characteristics of the single patient. In fact, the generally available sizes do not guarantee a uniform coverage either of the possible anthropometric range or of gender.
(b) The commercial prototypes in the state of art provide to implement robotic joints replicating the human ones, but the flexion carried out by the phalanges remains limited to the proximal one (between MCP and PIP, for long fingers, or between CMC and MCP, for the thumb) and to the intermediate one (between PIP and DIP, for long fingers, or between MCP and IP, for the thumb), by leaving the distal phalanx (between DIP and tip of the long finger, or between IP and tip of thumb) blocked, after the absence of the robotic joint (DIP and IP respectively for long fingers and thumb). This involves a limitation in the implementable total motion range (ROM) and a difference with the residual contralateral limb.
(c) Usually, the prosthetic devices of known art provide the production of single components and subsequently an assembly of the complete system, with consequent complexity of production/manufacturing nature and high costs.
(d) In order to govern actively the 21 GDL of the hand fingers independently an equal number of actuators is requested, with consequent mechanical complexity and high weight.

Zollo et al (“Biomechatronic Design and Control of an Anthropomorphic Artificial Hand for Prosthetic and Robotic Applications”, IEEE/ASME TRANSACTIONS ON MECHATRONICS, IEEE SERVICE CENTER, PISCATAWAY, NJ, US, vol. 12, no. 4, 1 Aug. 2007, pages 418-429) describes a method for designing an anthropomorphic hand prosthesis.

### SUMMARY OF THE INVENTION

The technical problem placed and solved by the present invention is then to provide a method for designing and manufacturing a hand prosthesis and a resulting prosthetic device, allowing to obviate the drawbacks mentioned above with reference to the known art.

Such problem is solved by a method according to claim 1.

Preferred features of the present invention are set forth by the depending claims.

The method according to the invention relates to the design and implementation of a poly-articulated prosthesis of customized hand for amputated patients at trans-radial level with healthy (that is not amputated) contralateral limb. The resulting prosthetic device meets both functional requirements, by assisting actively the patient with a low use complexity, and cosmetic requirements, to increase the acceptability of the device itself.

In particular, the method provides the main steps listed below, in sequence.
(i) Patient profiling, by means of registration of the kinematic trajectories of the fingers of the healthy contralateral hand and three-dimensional scan of the latter for a subsequent reproduction of the relative anthropometry and/or general aspect.
(ii) Kinematic synthesis of a virtual mechanism with 6 links and 7 rotative joints, each one of the latter with 1 GDL, for each finger. In particular, 3 links and 3 joints mime the kinematic chain of each finger of the hand, whereas the remaining 3 links and 4 joints perform kinematic coupling functions. The mechanism is synthetized through an analytical criterium of topological optimization based upon maximization of a performance index (GPI, Global Performance Index). This procedure takes into account the maximum proximity of the joint virtual trajectories to the recorded one of the minimization of the torque and/or actuation force required to balance a tip load.
(iii) Development of the physical device through additive manufacturing technologies. In particular, the proposed solution is characterized by mechanism topologically identical for each patient adopted in step (ii) and on a customized external interface, miming the anthropometry of the single patient as acquired in step (i). Such external interface can be implemented by means of covering shells shaped to the shape of the patient's individual fingers, then with a modular and not discrete customization (in sizes) as, on the contrary, in the state of art.

Said mechanism is synthetized through a numerical optimization process, which receives as input the anthropometric characteristics and the flexion-extension angles of the patient's joints obtained in step (i) and, preferably, the constraints associated with the manufacturing technology sub (iii) and identifies the kinematic solution closest to that of the subject.

The mechanism with 6 links and 7 joints provides an anatomy and a motion as close to human ones as possible and specific for the single patient, helping to increase the level of acceptability of the device.

Moreover, the use of kinematic couplings which reduce the number of GDL independent for each finger to one makes the system under-implementable, by allowing the use of one single actuator per finger. The under-implementation leads to a decrease in the design complexity, in the energy consumption, in weight and cost. Moreover, the actuator used to check the single kinematic chain can even be shared to govern several fingers.

The complexity and the costs for producing and assembling the customized devices are eliminated (even) through the use of technologies of additive manufacture based upon step (iii), according to a non-assembly approach. The latter provides to print not the single components of the device, but the entire assembly system, then already assembled except for the control electronics and for possible additional purely cosmetic portions.

Other advantages, features and use modes of the present invention will result evident from the following detailed description of some embodiments, shown by way of example and not for limitative purposes.

### BRIEF DESCRIPTION OF FIGURES

The figures of the enclosed drawings will be referred to, wherein:

FIG. 1 shows a scheme related to the steps for designing and implementing a prosthetic hand according to a preferred embodiment of the present invention;

FIG. 2 shows a preferred arrangement of 24 retroreflective passive markers affixed to the healthy hand contralateral to the amputated one, configured for acquiring kinematic trajectories of the fingers under various conditions of manipulation, motion and/or grasp according to the method of FIG. 1;

FIG. 3 shows a trajectory exemplifying the human joints PIP and DIP depending upon MCP acquired with the arrangement of FIG. 2; the solid line shows the real trajectory, whereas the dotted one shows the regression line; the image shows the initial and final instants used for the kinematic synthesis and the sloping values of the line;

FIG. 4 shows a preferred embodiment of a geometrical-topological scheme of the finger mechanism of long fingers (with three phalanges);

FIGS. 5A, 5B and 5C show, respectively, a kinematic scheme of the mechanism of FIG. 4 and two independent circuits thereof to describe the closing equations thereof;

FIGS. 6A and 6B show a design comparison between a preferred kinematic model (FIG. 6A) and a corresponding CAD model (FIG. 6B) of the single finger;

FIG. 7 shows preferred reference sections for the modelling the palm, according to a structure with four transverse plans;

FIG. 8 shows an example of prototype of customized prosthesis according to a preferred embodiment of the invention, from different angulations and in different motion configurations.

### DETAILED DESCRIPTION OF PREFERRED EMBODIMENTS

Various embodiments and variants of the invention will be described hereinafter and this with reference to the above-introduced figures.

Analogous components are designated in the different figures with the same reference number.

In the following detailed description, additional embodiments and variants with respect to embodiments and variants already treated in the same description will be illustrated limitedly to the differences with what already illustrated.

Moreover, the different embodiments and variants described hereinafter are likely to be used in combination, where compatible.

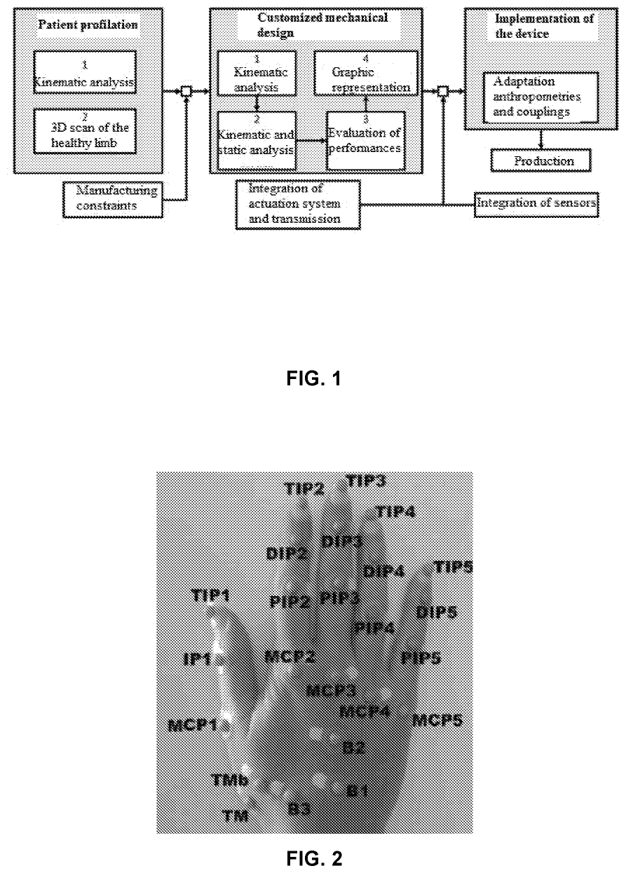

As mentioned in the preceding section and with reference to FIG. 1, the method for designing and manufacturing prostheses according to the invention is based upon the following main steps:
(i) patient profiling to obtain anatomical and functional parameters, by kinematic analysis of trajectories and 3D scan of the hand contralateral to the amputated one;
(ii) synthesis of a mechanical mechanism of the prosthesis, by analytical optimization of a planar mechanism with 1 degree of freedom (GdL) active for each finger, preferably by calculating a global performance index (GPI);
(iii) manufacturing of the physical device, through technologies of additive manufacturing.

Each one of such steps will be now described in greater detail with reference with preferred embodiments and variants.

### (i) Patient Profiling

### (i1) Kinematic Analysis

Based upon a preferred protocol, the patient is subjected to a functional clinical and psychological evaluation to verify if the criteria for applying the method for designing and manufacturing a hand prosthesis are met. Such criteria can be constituted by the following clinical outputs:

unilateral trans-radial amputation of hand;

hand contralateral to amputation without motor deficits and/or malformations;

length of the healthy contralateral hand preferably comprised between 173 and 205 mm (range defined based upon the anthropometric percentiles of adult men and women referred to in: [Pheasant, S., & Haslegrave, C. M. (2018). Bodyspace: Anthropometry, ergonomics and the design of work. CRC press]).

Based upon a preferred embodiment, instead, exclusion criteria are:
bilateral amputations, which would prevent the customization of the present step of the method;
in some embodiment variants, altered cognitive status (for example, the patient is not capable of understanding the declaration of consent or performing simple instructions such as opening and closing the healthy contralateral hand);
in some embodiment variants, presence of essential tremor.

The unilateral amputated patient is subjected to analysis of the healthy contralateral limb motion.

To the purpose of the kinematic analysis and with reference to FIG. 2, based upon a preferred embodiment variant on the healthy contralateral hand 24 retroreflective passive markers are applied according to the protocol indicated in [F. Cordella, L. Zollo, A. Salerno, D. Accoto, E. Guglielmelli, and B. Siciliano, “Human hand motion analysis and synthesis of optimal power grasps for a robotic hand,” International Journal of Advanced Robotic Systems, vol. 11, no. 3, p. 37, 2014].

The patient is preferably sitting comfortably with her/his elbow resting on a table placed in front of her/him and with the open hand (fingers extended in neutral position) and he/she is asked to repeat a free flexion-extension motion ten times, at first with the long fingers and then with the thumb. Such motor task is recorded by a motion analysis system, known on itself in the art, for the reconstruction of the 3D position of the markers and the extraction of the flexion-extension trajectories.

Still with reference to the arrangement shown in FIG. 2, the 3D position of the markers, originally expressed with respect to the global reference system of the cameras, is defined in a local reference system having origin in marker B1, axis x identified as verser \vec{B1B2}, axis y identified as verser generated by the vectorial product B1B3×B1B2 and axis z defined as \vec{x×y}. The coordinates of the generic marker Mi, with i=1, 2, . . . , 24, are expressed with respect to such local reference system by the coordinate transformation:

$${M}_{i}^{hand} = {R}_{cameras}^{hand} \cdot [ {M}_{i}^{cameras} - B {1}^{cameras} ] i = 1 , 2 , … , 24 ,$$

where,

$${M}_{i}^{hand}$$

represents the coordinates of the i-th marker expressed with respect to the base hand tern,

$${R}_{cameras}^{hand}$$

is the mutation matrix representing the orientation of the global tern of the cameras with respect to the base hand tern, and

$${R}_{i}^{cameras} and {B}_{1}^{cameras}$$

are respectively the coordinates of the i-th markers and of the marker B1 expressed with respect to the global reference system of the cameras.

The reference terns at the joints MCP of the long fingers and at the joints TM and MCP of the thumb are defined according to the kinematic protocol proposed in the above-mentioned publication. The flexion axes of the joints PIP and DIP of the long fingers and of the joint IP of the thumb, instead, are defined by the analysis of the main components (Principal Component Analysis, PCA), according to a process known in the art too. By concatenating vertically the coordinates of the markers positioned on each finger a matrix N×3 is obtained, wherein N is the number of samples and three the dimensions x, y, z of the markers (referred to the base tern).

By applying PCA to such matrix, three main components are obtained, the third one thereof (that is the direction along which the data show less variance) represents the flexion axis of the joints at issue. Once the flexion axes of each finger point are identified (that is, the axis z of the local tern at each joint), the axes x of the local terns at the joints are defined as the projections on the flexion plane of the versers joining the preceding marker at each joint with the marker positioned on the joint itself. At last, the axis y is defined by the vectorial product \vec{z×x}. For example, the axis x of the local tern at DIP is defined as the projection of the verser joining the markers PIP and DIP on the flexion plane detected by the axis z of the finger under examination, and the axis y is detected by the vectorial product \vec{z×x}.

The flexion angle of the joint is defined as Eulero angle which describes the tern rotation downstream of the joint with respect to the tern positioned on the joint itself. For calculating the couplings, only the flexion angles are of interest, that is the angles describing the tern rotation downstream with respect to the axis z of the interest joint. Once known the joint angles, the 10 motions performed by the subject are segmented. To this purpose, the ROM of each joint is calculated as θ_{MAX}−θ_{MIN}, where θ_{MAX} and θ_{MIN} are respectively the maximum and minimum angle recorded for a determined joint. The beginning of each flexion motion, for each finger, is segmented by detecting the time instant for which both following conditions are fulfilled:

$${θ}_{MCP} \geq 0$$

$${\dot{θ}}_{MCP} \geq 0.1 \cdot max ( {\dot{θ}}_{MCP} )$$

Where θ_{MPC} is the flexion angle of the joint MCP and θ_{MCP} is its first derivative. The end of the flexion motion, instead, is detected as the time instant thereat all joints of the finger under examination have reached an angle equal to 0.8 ROM.

The ten angular trajectories segmented for each point are resampled to the greater length and averaged between them to obtain an average angular trajectory for each point.

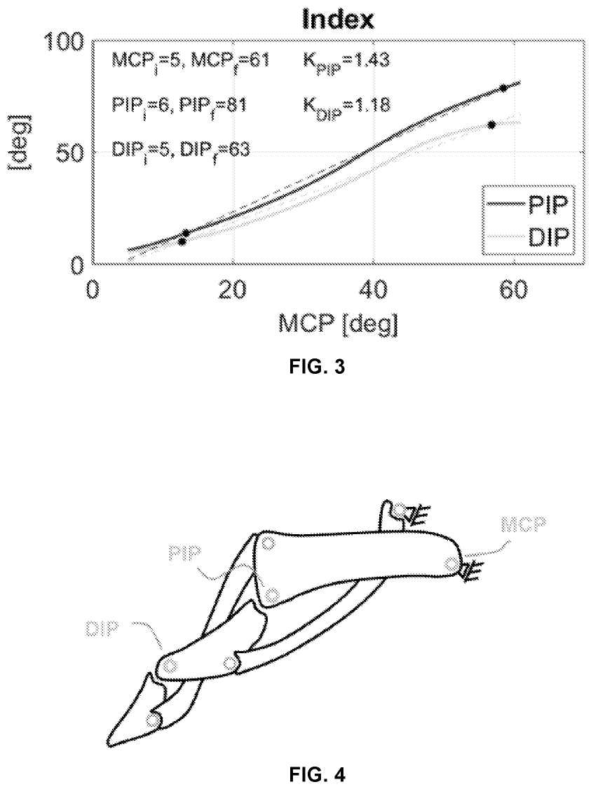

Starting from these average trajectories the inter-joint coupling relationships of the patient inside the single finger are extrapolated. In particular, the distal joints are represented as functions of the proximal joint (MCP for the long fingers, CMC for the thumb) and a linear regression is performed by extracting the slope of the regression line and intercepts it on the axis y. An analysis example for the index finger is shown in FIG. 3, which relates to the trajectory of the human joints PIP and DIP depending upon MCP. The solid line shows the real trajectory, whereas the dotted one shows the regression line. The image shows the initial and final instants used for the kinematic synthesis and the slope values K of the regression lines, which represent the coupling coefficients.

### (i2) Three-Dimensional Scan

Parallelly to the above-illustrated kinematic analysis a 3D scan of the healthy contralateral limb is performed, so as to be able to repeat the anthropometric characteristics thereof in the missing limb, in a mirrored manner.

Based upon a preferred protocol, the patient is invited to sit comfortably and to rest the arm on a support provided with a transparent base which allows to keep still the hand for few minutes and to scan it both in the palmar and dorsal face. Specifically, the patient is asked to rest the arm on the stiff portion of the support with the palm placed on a glass in order to allow a complete scan of the hand. The fingers, instead, are resting upon a thin beam support to reduce the areas in contact with the phalanges and avoid to modify excessively the shape thereof. In this position a trained operator scans the patient hand with 3D scanner. In this way a point cloud is obtained which is re-processed and transformed into a mesh through CAD software, known on itself in the art, to obtain a digital model of the hand of the single patient to be used as design input, but mirrored, in order to repeat indeed the missing limb.

### (ii) Customized Mechanical Planning (Kinematic Synthesis)

The kinematic, anthropometric and morphological data obtained in the previous step (i) can be used as input of a kinematic synthesis step performed automatically by electronic computer.

To such input data those related to the manufacturing constraints, for example tolerances between the pieces so that they can be reproduced with technologies of additive manufacturing, are added.

The process of mechanical design, meant as kinematic synthesis, is the same for each finger. Then, the case of a long finger will be described hereinafter, since it will speak about MCP, PIP and DIP, but the same type of analysis and design can be made for the thumb, by analysing CMC, MCP and IP, respectively.

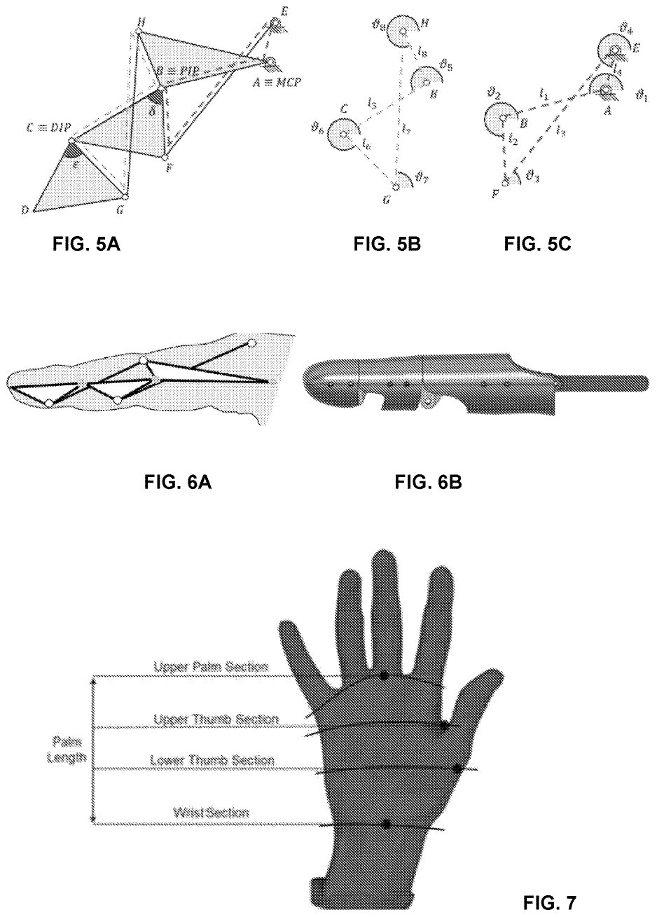

As shown in FIG. 4, the mechanism model of the single finger consists of 6 rigid members (including a member shown as frame) and 7 rotative joints with one degree of freedom, interconnected to form two quadrilaterals articulated in series and constituting a Watt mechanism. The latter is schematized even in FIG. 5A. It is divisible into two independent mechanical circuits, a proximal one shown in FIG. 5B and a distal one represented in FIG. 5C.

The proposed kinematic solution is characterized by a mechanism topologically identical for each patient (such as the one shown indeed in FIGS. 4 and 5A), which is synthetized through a numeral optimization process by receiving as input the anthropometric characteristics, the flexion-extension angles of the patient joints and, preferably, the constraints associated with the manufacturing technology.

Preferably, from a dimensional point of view the anthropometric characteristics are used as dimensional upper limit, so that the prosthetic finger is contained within the limits of the scanned human finger. The manufacturing constraints, instead, are used as lower limit, in particular one refers to the tolerance of the selected additive manufacturing technology and to the resolutive limits of the same. The flexion-extension couplings are extrapolated starting from the human kinematic trajectories, at an initial instant, in a natural configuration at rest, and a final instant, in a wholly flexed configuration, by recording two coupling terns MCP-PIP-DIP which will constitute the kinematic synthesis input. In particular, such conditions are those for which the human trajectory intersects the regression line, so that the prosthetic finger will perform a trajectory which surely interpoles the passage points selected in the healthy counterpart: somewhere else the kinematics will try to approximate the data of human points as much as possible.

The mechanism couples the 3 degrees of freedom of flexion-extension of the fingers by reducing them to one only degree of freedom by using the inter-joint relationships between the joints MCP, PIP and DIP for the long fingers and between CMC, MCP and IP for the thumb. The mechanism is synthetized kinetically in a discrete number of possibilities within the anthropometries granted by the finger under examination, through the method of the closing equations referred to in [Vinogradov, O. (2000). Fundamentals of kinematics and dynamics of machines and mechanisms. CRC Press].

With reference to FIGS. 5B and 5C, L_{ind}=j−l+1=7−6+1=2 results, where L_{ind} is the number of independent mechanical circuits (independent loop), j is the number of kinematic torques and/the number of members. For each one of such circuits a closing equation can be written:

$${l}_{1} \cdot {e}^{{iϑ}_{1}} + {l}_{2} \cdot {e}^{{iϑ}_{2}} + {l}_{3} \cdot {e}^{{iϑ}_{3}} + {l}_{4} \cdot {e}^{{iϑ}_{4}} = 0$$

$${l}_{5} \cdot {e}^{{iϑ}_{5}} + {l}_{6} \cdot {e}^{{iϑ}_{6}} + {l}_{7} \cdot {e}^{{iϑ}_{7}} + {l}_{8} \cdot {e}^{{iϑ}_{8}} = 0$$

where Iᵢ and θᵢ represent respectively the length of the i-th link and the goniometric circumference angle, taken counterclockwise, according to the Eulero notation. Such angles are expressed as function of the joint angles
∂_{MCP}, ∂_{PIP}, ∂_{DIP}
and of the geometrical contributions constituting the mechanism. The segments AB, BC, CD, represent the length of the phalanges, whereas the remaining geometric quantities constitute the unknown quantities of the kinematic problem.

We arrive, analytically, to two non-linear vectorial equations, one for each mechanical circuit, each one thereof provides two scalar equations, then two non-linear system of the type:

$$\underline{ϑ} ( {ϑ}_{MCP} , {ϑ}_{PIP} , {l}_{1} , {l}_{2} , {l}_{3} , {l}_{4} ) = 0$$

$$\underline{ψ} ( {ϑ}_{MCP} , {ϑ}_{PIP} , {ϑ}_{DIP} , {l}_{5} , {l}_{6} , {l}_{7} , {l}_{8} ) = 0$$

The analytical passage for the initial and final configurations described previously is imposed thereto. At last, the system is solved numerically.

The synthesis process of the kinematic structure is a recursive algorithm, known on itself in the art, which varies selectively the measurements of some mechanism members and calculates the analytical solution which, for each patient, guarantees the kinematics closest to the one of the recorded contralateral limb and which requires an implementation of the lowest binding reactions at the joints. In particular, six performance indexes are recorded which, weighed and added up, characterize a global performance index (GPI), dimensionless parameter varying from 0 and 1. GPI is so described analytically:

$$GPI ( k ) = {\sum }_{i} {w}_{i} {f}_{i}^{k} \qquad {GPI}_{best} = max GPI ( k )$$

The components of GPI, for the k-th mechanism, are described in Table 1 and, in the present embodiment, are one or more quantities selected in a group including: the Root-Mean-Square Error (RMSE) between human trajectory and mechanism trajectory (in terms of PIP and DIP); the deviation of the mean coupling of trajectories (PIP vs MCP, DIP vs MCP); the maximum actuating torque requested to balance a tip load and the maximum binding reactions at the joints, both normalized with respect to the maximum possible values. For calculating the last two indexes, the statics of the mechanism is solved by applying a load of 1N perpendicularly to the tip of the finger, that is the binding reactions and the unknown torque at a rotary joint upstream of the system are calculated. The analytical contributions shown in Table 1 are described hereinafter:

$${f}_{i}^{k}$$

i-th performance of the K-th mechanism (value between 0 and 1);
i=1, . . . , 6: number of performance indices;
k=1, . . . , N: number of possible real solutions (solution space of the closing equation);
wᵢ: i-th weight (value between 0 and 100%, equal to 20% for i=1, . . . , 4 and 10% for i=5, 6).

The mechanism which returns a better performance is the optimum one, that is the one with the highest GPI. Then, the kinematic solution will be obtained, which better approximates the human trajectory, which provides the minor binding reactions at the robotic joints and which requires a lower actuation torque to balance a tip load (most critical design condition).

After such evaluation, a sensitivity analysis is performed: the kinematic solutions are discarded which, due to small variations in parameters, can cause excessive performance reductions. In particular, the geometrical parameters of kinematic syntheses are made to change and the GPI is evaluated again after such variation. The sensitivity analysis was performed by calculating the total differential of the k-th mechanism, expressed as follows:

$$dGPI ( k ) = {\sum }_{i} \frac{\partial GPI ( k )}{\partial {ℓ}_{i}} d {ℓ}_{i}$$

The optimum mechanism is the one which is obtained by evaluating the minimum of the total differential of GPI, that is mind GPI (k).

A preview of the trajectory of the optimum kinematics, of the loads and of the inter-joint couplings is provided to the designer.

TABLE 1
Global performance index (GPI) and components thereof

| | |
|---|---|
| COMPONENT | DEFINITION |

KINEMATIC PERFORMANCE

| | |
|---|---|
| PIP linear trend | f₁ = RMSE_{PIP} |
| PIP deviation from human coupling | ${f}_{2} = 1 - \frac{\vert {K}_{PIP , robot} - {K}_{PIP , human} \vert}{{K}_{PIP , human}}$ |
| DIP linear trend | f₃ = RMSE_{DIP} |
| DIP deviation from human coupling | ${f}_{4} = 1 - \frac{\vert {K}_{DIP , robot} - {K}_{DIP , human} \vert}{{K}_{DIP , human}}$ |

STATIC PERFORMANCE

| | |
|---|---|
| Actuation torque | ${f}_{5} = \frac{max τ ( {ϑ}_{MCP} )}{{max}_{k} max τ ( {ϑ}_{MCP} )}$ |
| Reaction forces | ${f}_{6} = \frac{{max}_{j} {F}_{j}}{{max}_{k} {max}_{j} {F}_{j}}$ |

* * *

(iii) Implementation of the Prosthetic Device (Production by Means of Additive Manufacturing)

The parametric CAD models of the finger are adapted based upon the output of the preceding step (ii), which provides as output the optimum sizes of the link. The shape (cosmetic aspect) is derived from the results of step (i). In detail, in order to obtain the shape of each finger the profiles and the guiding lines are identified as follows: the profiles are identified by sectioning the finger scan with a transverse plane at each anatomical joint. The so-obtained section is approximated to an ellipse with axes corresponding to width and thickness of the finger on the section plane. The guiding lines are obtained by the projection of the finger on the sagittal and coronal planes.

FIGS. 6A and 6B indeed provide a design comparison between kinematic model (FIG. 6A) and model CAD (FIG. 6B) of the single finger.

The three phalanges are perforated to house the trees corresponding to the human joints. The two connecting links, which reduce the degrees of freedom of the kinematic chain to one, are at last modelled. Lastly, one verifies: the robustness of each finger through criterium of von Mises and the absence of interferences between the links in the considered motion range. Analogously, the palm is reconstructed starting from its scanning by sectioning it with 4 transverse planes, passing through the considerable points shown in FIG. 7, which are at the height of the wrist, above and under the thumb and on the digital-palmar crease of the middle finger. The so-defined profiles are connected by guiding lines obtained, even in this case, by projecting the palm on sagittal and coronal plane. The so-obtained palm is dug internally to house electronics and motion transmission system.

The costs for producing and assembling the customized device are reduced by using technologies of additive manufacturing. In particular, the assemblies of the single fingers will be printed, whereas the palm will be printed separately to allow the electronic systems to be able to be added without problems.

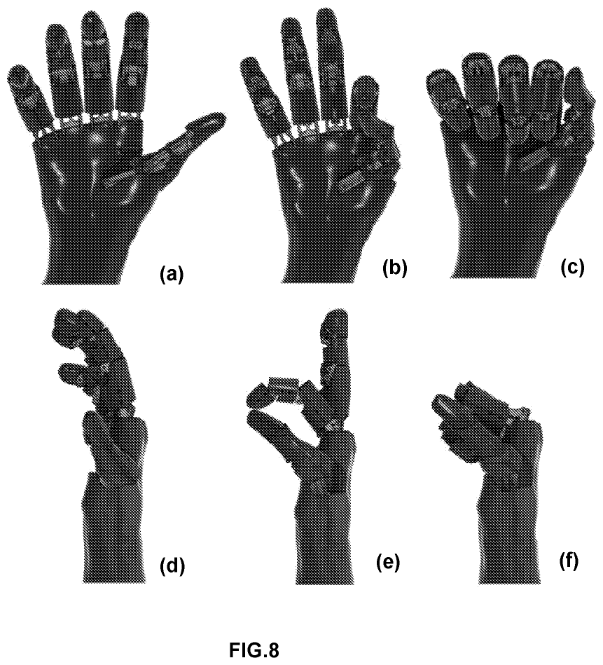

FIG. 8 shows the CAD model of the hand prosthesis designed according to the herein described embodiment. By way of example, Table 2 hereinafter shows the weight of a prosthesis implemented with Nylon12 (PA12), biocompatible material, widely used in prosthetic applications. Depending upon the length (HL) and the width (HB) of hand, the overall weight remains contained and well below 600 g, average weight of an adult hand.

TABLE 2
Weight of the mechanical structure (palm and 5 fingers)
of two hand prosthesis implemented in PA12.

| | Weight of | | | | | Weight | Total |
|---|---|---|---|---|---|---|---|
| | prosthetic fingers [g] | | | | | of palm | weight |
| HL [cm]:HB [cm] | I | II | III | IV | V | [g] | [g] |
| 16.7:7.3 | 8.9 | 13.7 | 15.7 | 13.2 | 15.3 | 103.1 | 169.9 |
| 18.0:8.0 | 11.4 | 17.5 | 20.1 | 16.9 | 19.6 | 124.4 | 209.9 |

The mechanical structure is arranged for the integration with commercial systems for implementation (for example DC brushless motors, servomotors, stepper motors) and transmission of the motion both with stiff (for example planetary gear motor, stiff links, other) and yielding (cable-pulley systems and/or rocker arms, yielding links, other) transmission.

The system check can be implemented through commercial surface myoelectric interfaces.

Since the single prosthetic fingers are characterized by one single GdL, it is sufficient to insert one single actuator to govern the whole kinematics of each finger.

By pure way of example, an embodiment example is herein reported, which provides the use of a rotary actuator, for the F/E of the four long fingers and a combined motion of F/E and A/A for the thumb.

The use of a 9 W motor Faulhaber 2214006BXTR Ø 22 mm, brushless, is provided, whose characteristics are reported hereinafter:
No load speed=5740 rpm
Nominal speed=1200 rpm
Nominal torque=9.5 mNm
Max. efficiency=72%
Max. speed=10000 rpm
Max. radial load (5 mm from flange)=6 N
Weight=25.5 g
Length=26.2 mm
Diameter=22 mm

By pure way of example, another embodiment example is herein reported, which provides the use of two rotary actuators, one for F/E of the five fingers and one for A/A of the thumb.

For each GdL the use of a 1.5 W motor Maxon RE 10 Ø 10 mm, brushless, is provided, whose characteristics are reported hereinafter:
No load speed=12800 rpm
Nominal speed=6530 rpm
Nominal torque=1.5 mNm
Max. efficiency=76%
Max. speed=19000 rpm
Max. radial load (4 mm from flange)=0.4 N
Weight=10 g
Length=34.9 mm
Diameter=10 mm

The actuation module can be connected to a motion transmission system based upon cables and pulleys which allows to transfer the torque to each finger and to manage the interaction with the objects when this is partial (that is when not all fingers come contemporarily in contact with the object).

The present invention has been described so far with reference to preferred embodiments. It is to be meant that any embodiments belonging to the same inventive core may exist, as defined by the protective scope of the herebelow reported claims.

### Glossary and Acronyms

Kinematic synthesis: creating the geometry and the structure of a mechanism to implement assigned motion laws.
CAD: Computer-Aided Design.
ROM: Range of Motion. It is the width of the motion which a joint can perform in the space, that is the degree of excursion which it can preform along its complete range of motion, both it is active or passive.
ADL: Activities of Daily living. They are the activities, thereamong eating, drinking, taking care of the person, ambulating, common in an individual's daily life.
GDL: degrees of freedom. It is the number of independent variables necessary to determine univocally the position of a point in the space and it is equal to the number of coordinates necessary to describe its motion inside the space of the possible configurations.
MCP: MetaCarpoPhalangeal joint. In the long fingers, it is the connecting joint between metacarpus and proximal phalanx, whereas in the thumb it connects proximal and distal phalanges.
PIP: Proximal InterPhalangeal joint. It is only in the long fingers and connects proximal and intermediate phalanges
DIP: Distal InterPhalangeal joint. It is only in the long fingers and connects intermediate and distal phalanges.
IP: InterPhalangeal joint). It is only in the thumb and connects intermediate and distal phalanges.
CMC: Carpo-MetaCarpal joint. It connects the metacarpal phalanx to the carpus. It is present in all five fingers and, in case of the thumb, it is also called TM (TMC) or TrapezioMetaCarpal joint.
GPI: Global Performance Index. It is the value used in the present description to define the kinematic and static optimization of the finger mechanism.
RMSE: Root-Mean-Square Error. It designates the mean square discrepancy between the values of the observed data and the values of the estimated data. In this case the observed data correspond to the mechanism trajectory, whereas the estimated ones correspond to the wished human trajectory.

1. A method for designing and manufacturing a customized hand prosthesis with one or more fingers, including the following steps:
(i) patient profiling by means of: (i1) kinematic analysis of trajectories of the finger(s) of the healthy hand contralateral to the one to be prosthesised; and (i2) three-dimensional scan of the healthy contralateral hand;
(ii) computer-executed kinematic synthesis of a joint and link configuration of the prosthesis, based, for each finger, on a topologically virtual mechanism model identical for each subject and for each finger, which mechanism model comprises 6 links and 7 rotative joints, each one of the latter with 1 degree of freedom, wherein a kinematic chain with 3 links and 3 joints mimics the finger, while other 3 links and 4 joints perform kinematic coupling functions; said kinematic synthesis step being performed by means of an optimization algorithm which receives as input anthropometric data and flexion-extension angles of the subject's finger joints acquired in said step (i) and outputs a virtual mechanism configuration selected by minimizing the difference between trajectories of virtual joints and trajectories measured in said step (i); and
(iii) manufacturing of the hand prosthesis according to the virtual mechanism configuration of said step (ii), by means of additive manufacturing, in particular 3D printing.

2. The method according to claim 1, wherein said virtual mechanism model simulates the metacarpophalangeal (MCP), proximal interphalangeal (PIP) and distal interphalangeal (DIP) joints for each long finger and the carpometacarpal (CMC), metacarpophalangeal (MCP) and interphalangeal (IP) joints for the thumb.

3. The method according to claim 1, wherein said virtual mechanism model comprises, for each finger, 6 rigid members and 7 rotative joints interconnected to form two quadrilaterals articulated in series constituting a Watt mechanism.

4. The method according to claim 1, wherein said optimization algorithm is of the recursive type.

5. The method according to claim 1, wherein said optimization algorithm performs a minimization of the torque and/or actuating force of each joint to balance a tip load.

6. The method according to claim 1, wherein said optimization algorithm receives as input parameters relating to constraints associated with the manufacturing technology used in said step (iii).

7. The method according to claim 1, wherein said optimization algorithm calculates a global performance index (GPI), wherein said global performance index is calculated as follows:
$$GPI ( k ) = {\sum }_{i} {w}_{i} {f}_{i}^{k} \qquad {GPI}_{best} = max GPI ( k )$$
wherein k identifies a finger mechanism and
$${f}_{i}^{k}$$
i-th performance of the k-th mechanism (value between 0 and 1);
i=1, . . . , 6: number of performance indices;
k=1, . . . , N: number of possible real solutions (solution space of a closure equation);
wᵢ: i-th weight (value between 0 and 100%, equal to 20% for i=1, . . . , 4 and 10% for i=5, 6),
wherein f is a function selected in a group comprising: Root-Mean-Square Error (RMSE) between the trajectory obtained in step (i1) and the trajectory of the virtual mechanism; deviation of the mean coupling between the trajectory obtained in step (i1) and trajectory of the virtual mechanism; maximum actuating torque required to balance a peak load; maximum binding reaction at the joints.

8. The method according to claim 1, wherein in said step (ii) the number of independent degrees of freedom for each finger is reduced to and in said step (iii) a single actuator per finger is used, with the possibility of actuating with the same actuator even more fingers.

9. The method according to claim 8, wherein, in said step (ii), the input angular trajectories for each joint are resampled to the greater length and averaged between them to obtain an average angular trajectory for each point and wherein starting from said average trajectories inter-joint coupling relationships are extrapolated for each finger; in particular, the distal joints are represented as functions of the proximal joint, in particular the metacarpophalangeal joint for the long fingers, the carpometacarpal joint for the thumb, and a linear regression is performed by extracting the slope of the regression line which represents the coupling efficient.
